(19) United States
(12) Patent Application Publication
(10) Pub. No.: US 2014/0303503 A1
(43) Pub. Date: Oct. 9, 2014

(54) APPARATUS AND METHOD FOR DETERMINING RESPIRATION SIGNALS FROM A SUBJECT

(71) Applicant: KONINKLIJKE PHILIPS N.V., Eindhoven (NL)

(72) Inventors: Mukul Julius ROCQUE, Eindhoven (NL); Jens MUEHLSTEFF, Aachen (DE)

(73) Assignee: KONINKLIJKE PHILIPS N.V., EINDHOVEN (NL)

(21) Appl. No.: 14/228,349

(22) Filed: Mar. 28, 2014

Related U.S. Application Data
(60) Provisional application No. 61/809,964, filed on Apr. 9, 2013.

(30) Foreign Application Priority Data
Apr. 9, 2013 (EP) 13162887.7

(51) Int. Cl. A61B 5/08; A61B 5/00

(52) U.S. Cl. CPC A61B 5/0816; A61B 5/0075
USPC 600/476; 600/407

(57) ABSTRACT

An apparatus and a method for determining respiration signals from a subject are disclosed. The apparatus comprises a receiving unit for receiving image data determined from the subject in a field of view, a processing unit for evaluating the image data, wherein the processing unit is adapted to determine a plurality of different alternating signals corresponding to vital sign information of the subject from a plurality of different areas of the field of view on the basis of movement pattern, and an evaluation unit for evaluating the different alternating signals and for determining a plurality of different respiration signals from the subject on the basis of the different alternating signals determined from the different areas of the field of view.

### CROSS REFERENCE TO RELATED APPLICATIONS

This application claims the benefit of U.S. provisional application Ser. No. 61/809,964 filed Apr. 9, 2013 and European provisional application serial no. 13162887.7 filed Apr. 9, 2013, both of which are incorporated herein by reference.

### FIELD OF THE INVENTION

The present invention relates to an apparatus and a method for determining respiration signals from a subject, wherein image data determined from the subject in a field of view is received and the respiration signals are determined on the basis of movement pattern determined in the image data.

### BACKGROUND OF THE INVENTION

Vital signals of a subject or a patient and in particular the respiration rate of a subject can be monitored remotely using contactless sensors such as a video camera. A general method for determining a respiration rate from image data by means of pattern detection is known e.g. from WO 2012/140531 A1. Since the subject to be measured can be located freely in the field of view of the camera and since the relevant area from which the vital signs should be derived can be located freely in the field of view of the camera, the subject and the relevant area have to be detected and defined for extraction of the desired vital sign information such as the respiration rate of the subject. Further, different movement pattern indicative for vital sign information and not indicative for vital sign information have to be identified and distinguished for a precise remote measurement of the vital sign information.

The traditional identification of the region of interest in general is based on detection of human being, e.g. the face or the chest or by using background segmentation. For identification of a human being and for measuring the vital signs from the remote image detection measurement such as a pulse or a respiration rate from a region of interest, US 2009/0141124 suggests to detect the contour of an infrared video segment to select the region of interest representing a portion of the subject to be measured.

Further, WO 2012/093320 A2 discloses a video detection device for detecting vital sign information from a subject, in particular photo-plethysmography signals from the subject, wherein the video data is divided in different blocks in order to select a region of interest which is in this case the skin of the subject in order to determine the vital sign information automatically in the field of view.

The traditional method for measuring the respiration is the inductive plethysmography wherein the respiration is detected by a breathing band measuring changes in the chest or abdomen cross-sectional area by placing a wire turn around the torso of the subject. Typically two breathing bands are used in order to distinguish thoracic and abdominal breathing. To measure the respiration of the subject precisely and to identify special injuries or paralysis, the independent measurement of the thoracic and abdominal breathing is necessary.

The disadvantage of the known methods for measuring respiration signals from a subject is that only one respiration signal can be determined remotely from the subject wherein only a coarse respiration analysis is possible or that the systems which measure precisely different respiration signals from the subject are uncomfortable for the user due to the use of contact measurement sensors.

### SUMMARY OF THE INVENTION

It is an object of the present invention to provide an improved apparatus and a corresponding improved method for determining respiration signals from a subject which is more precise and more comfortable for the user.

According to one aspect of the present invention, an apparatus for determining respiration signals from a subject is provided, comprising:
a receiving unit that receives image data determined from the subject in a field of view,
a processing unit that evaluates the image data, wherein the processing unit is adapted to determine a plurality of different alternating signals corresponding to vital sign information of the subject from a plurality of different areas of the field of view on the basis of movement pattern, and
an evaluation unit that evaluates the different alternating signals and that determines a plurality of different respiration signals from the subject on the basis of the different alternating signals determined from the different areas of the field of view.

According to another aspect of the present invention a method for determining respiration signals from a subject is provided, comprising the steps of:
receiving image data determined from the subject in a field of view,
evaluating the image data,
determining a plurality of different alternating signals corresponding to vital sign information of the subject from a plurality of different areas of the field of view on the basis of movement pattern,
evaluating the different alternating signals, and
determining a plurality of different respiration signals from the subject on the basis of the different alternating signals determined from the different areas of the field of view.

According to still another aspect of the present invention, a computer readable non-transitory medium is provided having instructions stored thereon which, when carried out on a computer, cause the computer to perform the steps of the method according to the present invention.

The present invention is based on the idea to measure different respiration signals from one subject on the basis of a contactless measurement and to provide an improved and precise respiration measurement which is comfortable due to the contactless measurement for the user. The different respiration signals are determined on the basis of movement pattern determined from image data captured from the subject to be measured, wherein the movement pattern of different areas in the field of view are used to determine the different respiration signals. Hence, the movement of different portions of the subject corresponding to the respiration of the subject can be determined independently such that e.g. the thoracic and abdominal breathing can be determined independently and comfortable for the user so that the whole breathing information can be determined with low technical effort. On the basis of the different breathing signals, additional diagnostics can be performed so that the vital sign detection becomes more precise.

Preferred embodiments of the present invention are defined in the dependent claims. It should be understood that the claimed method has similar and/or identical preferred embodiments as the claimed apparatus and as defined in the dependent claims.

In a preferred embodiment, the processing unit is adapted to define a plurality of image sections in the image data and to determine one alternating signal corresponding to the vital sign information from each of the image sections on the basis of movement pattern detection. This is a possibility to identify the vital sign information from the whole field of view with low technical effort.

In a preferred embodiment, the processing unit is adapted to define the different image sections as an array of image sections in the image data. This is a simple solution to analyze the whole image data and to analyze the whole field of view in order to determine the different vital sign information of the subject.

In a preferred embodiment, the apparatus further comprises a frequency analysis unit for determining spectral parameter of the alternating signals determined from the different image sections and a selection unit for selecting different image sections on the basis of the spectral parameter as the different areas to determine the different respiration signals. This is a reliable possibility to determine different regions of interest in the field of view from which vital sign information can be derived.

In a preferred embodiment, the spectral parameter determined from the different image sections is a spectral energy of the alternating signals. This is a possibility to distinguish vital sign information from disturbing signals and noise with high reliability.

In a preferred embodiment, the selection unit is adapted to select the image sections if the spectral energy of a predefined frequency band of the alternating signals exceeds a threshold level. This is a possibility to analyze the spectral parameter with low technical effort.

In a preferred embodiment, the different respiration signals are determined on the basis of motion vectors derived from different portions of the subject. By means of the motion vector derived from different portions of the subject, the different respiration signals corresponding to e.g. thoracic and abdominal respiration can be determined.

In a preferred embodiment, the different respiration signals are time-dependent alternating signals having different waveforms. This is a possibility to determine additional diagnostic information from the subject in addition to the simple respiration rate.

In a preferred embodiment, the different respiration signals are time-dependent alternating signals having a phase shift to each other. This is a possibility to distinguish different respiration signals of the subject in order to determine additional diagnostic information.

In a preferred embodiment, the evaluation unit is adapted to determine a signal difference of the different respiration signals as additional respiration information from the subject. This is a solution to automatically determine additional respiration information beyond the respiration rate for additional diagnostics.

In a preferred embodiment, the evaluation unit is adapted to determine the phase shift of the different respiration signals and to combine the different respiration signals to one general respiration signal considering the determined phase shift. This is a possibility to determine a single respiration signal having an increased preciseness and a higher reliability.

In a further preferred embodiment, the evaluation unit is adapted to determine an array of respiration signals on the basis of the different respiration signals derived from the different image sections to provide a spatial respiration map of the subject. This is a possibility to determine the whole respiration information from the subject in order to provide additional diagnostic possibilities.

In a further preferred embodiment, the selection unit is adapted to determine a weight factor for each of the selected different image sections and wherein the evaluation unit is adapted to determine the different respiration signals on the basis of the alternating signals of selected image sections weighed by means of the respective weight factor. This is a possibility to consider a signal strength of the alternating signals in order to increase the preciseness of the determined respiration signal, since disturbing signals or noisy signals are less considered than those signals which have a high strength.

It is further preferred if the selection unit is adapted to perform the selection on a regular basis and wherein the weight factor for each of the selected image sections is determined on the basis of a frequency of selection of the respective image section. This is a possibility to determine the signal strength and the weight factor with low technical effort.

As mentioned above, the present invention provides a possibility to determine different vital sign information from one subject on the basis of contactless remote measurements by using image data determined from a field of view including the subject to be measured. Since the alternating signals are determined on the basis of movement pattern determined from different areas of the field of view, respiration signals from different portions of the subject, e.g. the thorax and the abdomen can be determined corresponding to different respiration techniques in order to increase the preciseness of the respiration detection and to determine additional information from the respiration of the subject. Hence, additional diagnostics can be performed and the detection of the respiration has a higher reliability and is more precise and can be determined comfortable on the basis of contactless measurements.

In still another aspect of the present invention an apparatus for determining respiration signals from a subject is presented, comprising:
a receiving unit that receives image data determined from the subject in a field of view,
a processing unit that defines a plurality of image sections in the image data and that determines one alternating signal corresponding to the vital sign information from each of the image sections on the basis of movement pattern detection, wherein the processing unit is adapted to determine the alternating signals corresponding to the vital sign information of the subject from different image sections of the field of view on the basis of movement pattern, and
an evaluation unit that evaluates the different alternating signals and that determines a plurality of different respiration signals on the basis of the movement pattern derived from different portions of the subject and the different alternating signals determined from the different areas of the field of view.

### BRIEF DESCRIPTION OF THE DRAWINGS

These and other aspects of the invention will be apparent from and elucidated with reference to the embodiment(s) described hereinafter. In the following drawings

FIG. 1 shows a schematic illustration of a general layout of an apparatus for determining respiration signals from a subject,

FIG. 2 shows a schematic illustration of a subject's motion indicative of respiration signals,

FIG. 3 shows a timing diagram of an alternating signal derived from the subject,

FIG. 4 shows a frequency diagram of the alternating signal shown in FIG. 3,

FIG. 5 shows a schematic image segmentation for illustrating the detection of the different alternating signals in the field of view,

FIG. 6 shows three respiration signals determined from different portions of the field of view and the respective images from which the respiration signals are determined, and

FIG. 7 shows a schematic block diagram representing the steps of an embodiment of a method to determine respiration signals from a subject in a field of view.

### DETAILED DESCRIPTION OF THE INVENTION

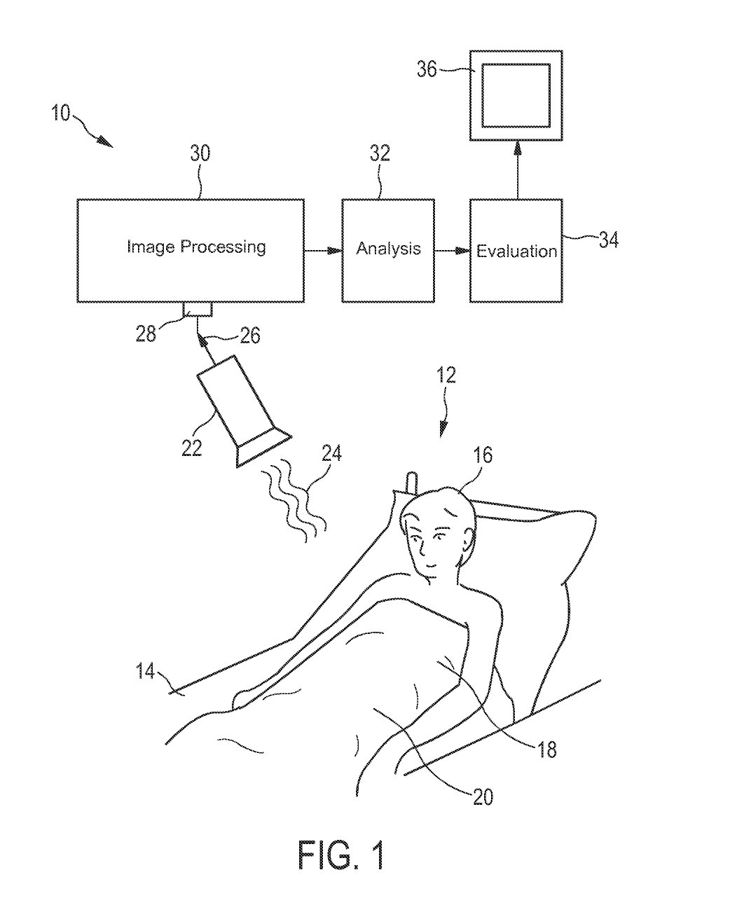

FIG. 1 shows a schematic drawing of an apparatus generally denoted by 10 for determining respiration signals from a subject 12. The subject 12, e.g. a patient staying in bed is resting on a support 14. The subject's head 16 is usually a non-indicative portion regarding the respiration of the subject 12, wherein the chest 18 or the thorax 18 and the belly 20 or the abdomen 20 are indicative portions regarding the respiration of the subject 12. The general problem is that different respiration signals corresponding to thoracic and abdominal respiration cannot be measured independently and contactless precisely with low technical effort. Usually, merely the respiration rate or the heartrate are detected by means of camera systems or remote systems in general.

The apparatus 10 is connected to an image detection device 22, e.g. a monochromatic camera which can be used for recording image frames of the subject 12. The image frames can be derived from electromagnetic radiation 24 emitted or reflected by the subject 12. For extracting the image information from the image data, e.g. a sequence of image frames, the image detection device 22 is connected via an interface 28 to an image processing unit 30. The image detection device 22 may be part of the apparatus 10 or may be an external camera 22 such that the image data 26 is merely provided to the interface 28 in order to provide the image data 26 to the apparatus 10 in general.

The image detection 22 is adapted to capture images belonging to at least a spectral component of the electromagnetic radiation 24. The image detection device 22 may provide continuous image data or a discrete sequence of image frames captured from a field of view including the subject 12 to be measured.

The image processing unit 30 is adapted to receive the image data 26 from the image detection device 22 via the interface 28, to evaluate the image data 26 in general and to detect different regions of interest of the subject 12, e. g. the thorax 18 and the abdomen 20 as indicative portions of the respiration of the subject 12. In order to detect the region of interest, e.g. the thorax 18 and/or the abdomen 20, the image processing unit 30 is adapted to divide the captured images in sections or areas of the field of view and to evaluate the image sections separately in order to determine the region of interest. The image processing unit 30 divides the captured images into the image sections and detects motion vectors from the different sections corresponding to the motion of the subject in the field of view including the motion of the thorax region 18 and/or the abdomen region 20 of the subject 12 as indicative portions of the respiration. The motion vectors are determined by means of pattern detection in the image sections or by means of edge detection in the image sections. A method for edge or pattern detection and for deriving the motion vectors from the captured image frames is for example disclosed by WO 2012/140531 A1.

The imaging processing unit 30 is connected to an analysis unit 32. The image processing unit 30 determines alternating signals from the motion vectors from each of the image sections and provides the alternating signals to the analysis unit 32.

The analysis unit 32 determines a spectral parameter of each of the alternating signals by means of a frequency analysis unit included in the analysis unit 32 as described in detail in the following. The spectral parameter of each of the sections in the image data 26 are analyzed by a selection unit which is part of the analysis unit 32. The selection unit selects those sections of the image data from which an alternating signal is derived which is supposed to correspond to a respiration signal. The selection unit selects the sections on the basis of the respective spectral parameter. The spectral parameter is a frequency spectrum or a spectral energy distribution of each of the alternating signal. Since the respiration signal of the subject has a characteristic spectral energy distribution or a characteristic frequency, the selection unit can select the sections which comprise the respiration signals of the subject 12, and, therefore, the selection unit identifies the thorax 18 and/or the abdomen 20 of the subject 12 in the image data 26 for determining different respiration signals.

The selection unit also determines a weight factor for each of the different image sections dependent on the frequency analysis as described in the following. The weight factor is in general dependent on the frequency how often each of the image section is selected. Hence, the weight factor represents a factor corresponding to a signal strength of the alternating signals so that the respective alternating signal from each of the image sections can be considered according to the signal quality.

The analysis unit 32 is connected to an evaluation unit 34 and provides the alternating signals to the evaluation unit 34 for determining respiration signals corresponding to the respiration of the subject 12. The evaluation unit 34 receives the alternating signals determined from the different image sections and the respective weight factors for the different image sections from the analysis unit 32 and calculates the different respiration signals on the basis of the alternating signals, the weight factors and the different regions from which the alternating signals are derived. Hence, the respiration signals are calculated on the basis of the image data 26 and can be determined entirely contactless, wherein the respiration signals can be derived independently from different portions, e.g. the thorax 18 and the abdomen 20 of the subject 12.

The so-calculated respiration signals can be provided to a display 36 to display the measured respiration signals continuously or frequently.

Hence, the thoracic and abdominal breathing can be determined entirely contactless and independently from each other so that the respiration measurement becomes more precise and additional information can be derived from the respiration of the subject 12 in order to diagnose additional injuries such as spinal cord injuries or diaphragmic paralysis.

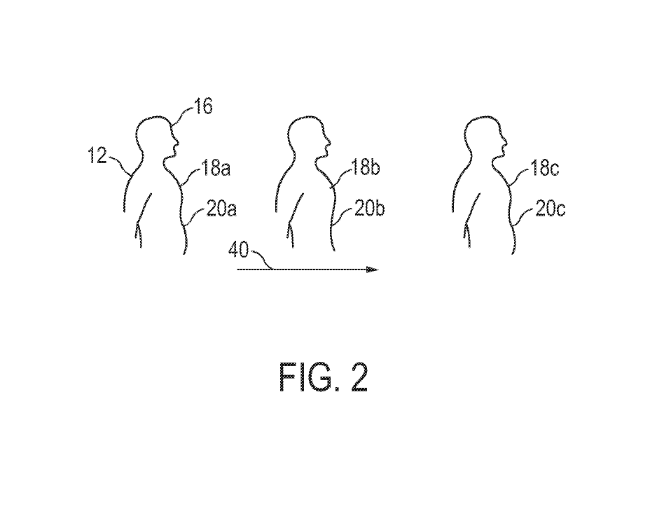

FIG. 2 shows a schematic illustration of the subject 12 in order to describe the remote measurement of the respiration of the subject 12. The subject 12 undergoes a characteristic motion of a first indicative portion 18 (the thorax 18) and a second indicative portion 20 (the abdomen 20) due to the respiration. When breathing, an expansion and a contraction of the lungs causes slight motion of the two indicative portions 18, 20, i.e. lifting and lowering the thorax 18 and the abdomen 20. Usually, the thorax 18 and the abdomen 20 are lifting and lowering in an alternating fashion such that the thorax 18 is lifting while the abdomen 20 is lowering and vice versa.

Over time as indicated by an arrow 40, the indicative portions 18, 20 are moved between a contracted position indicated by reference numerals 18a, 20b and 18c and an extracted position indicated by 20a, 18b and 20c. Essentially, based on the motion pattern, for instance the respiration rate or the respiration rate variability or the respiration volume can be assessed by means of pattern or edge detection in the captured image sequence. While the indicative portions 18, 20 are pulsating over time, the head 16 as a non-indicative portions remains substantially motionless. It should be understood that the thorax 18 and the abdomen 20 are examples as indicative portions for the respiration and that also other portions of the subject 12 can be detected in order to determine additional respiration signal such as movements at the lower rib of the subject 12.

Certainly, also the head 16 undergoes diverse motion over time. However, these motions do not correspond to the periodic pulsation of the thorax 18 or the abdomen 20 and can be distinguished by means of the frequency analysis unit.

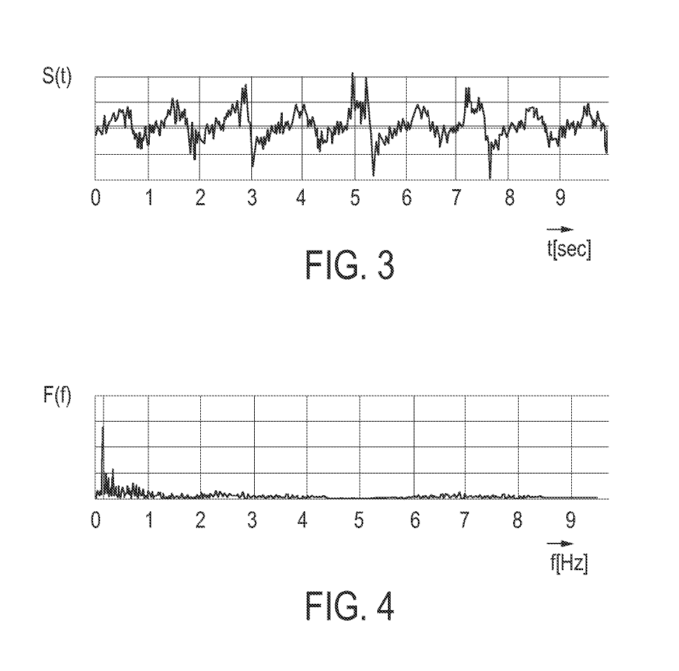

FIG. 3 shows a timing diagram of an alternating signal derived from the movement pattern and/or from motion vectors of the different image sections which can be for example determined on the basis of a frame or an edge detection in the respective image section. The alternating signal is generally denoted by S(t). The alternating signal S in this particular case corresponds to the movement of the thorax 18 or the abdomen 20 of the subject 12 derived from an image section corresponding to the image data received from the respective indicative portion 18, 20. The alternating signal S shows a characteristic variation corresponding to the movement of the chest 18 or the abdomen 20, i.e. the breathing of the subject 12. The alternating signal S also shows a high-frequency noise superimposed to the breathing.

The alternating signals S are derived from each of the image sections of the field of view wherein a plurality of image sections comprise vital sign information such as a breathing rate and many image sections may comprise disturbing signals which are not related to vital sign information of the subject 12 or other alternating signals which comprise mostly high-frequency noise. In order to identify those image sections from which vital sign information can be derived, the analysis unit 32 comprises the frequency analysis device to perform a frequency analysis of the alternating signals S. The frequency analysis is preferably performed by filtering the alternating signals S and/or by performing a Fourier Transformation, in particular a Fast Fourier Transformation (FFT) of the alternating signal S. From the alternating signals S, a frequency spectrum is derived in order to identify the image section including vital sign information corresponding to the respiration of the subject 12 as described in the following.

FIG. 4 shows a frequency spectrum of the alternating signal S shown in FIG. 3 generally denoted by F(f). The frequency spectrum F shows a large frequency component in a low frequency band, in this particular case between 0 and 1 Hertz, which correspond to the breathing rate of an adult which is normally not higher than 1 Hertz, i.e. 60 breathes per minute. The frequency components higher than a predefined frequency band, e.g. 1 Hertz for adults and 2 Hertz for infants are usually disturbing signals in the image data 26 or correspond to noise of the alternating signal S. In order to characterize the quality of the alternating signal S, the spectral energy of the alternating signal S is determined and an image section is defined as an image section including vital sign information if the spectral energy of the alternating signal S in a predefined frequency band exceeds a predefined threshold level or exceeds a percentage of spectral energy compared to a second frequency band, e.g. the whole frequency spectrum. E.g. if the spectral energy between 0 and 1 or 2 Hertz is larger than a predefined threshold level, e.g. larger than 50% of the entire spectral energy of the alternating signal S or a predefined range of the spectrum, e.g. 2 . . . 3 Hz, 3 . . . 4 Hz, . . . On the basis of the spectral energy, the image sections are selected in the field of view and to determine the region of interest as described in the following and to determine the different respiration signals.

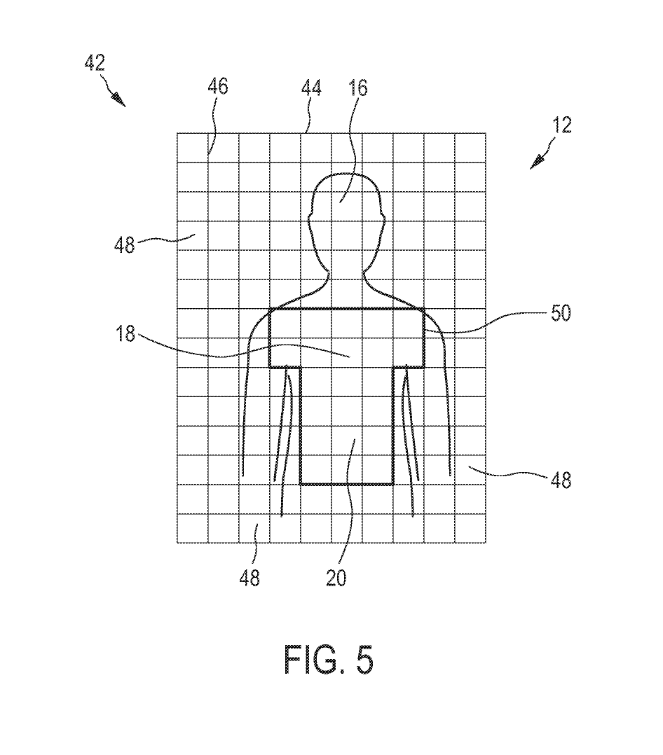

FIG. 5 shows a schematic image from a field of view for explaining the detection of the different respiration signals from the subject 12 on the basis of detected image data 26. The field of view detected by the image detection device 22 shown in FIG. 5 is generally denoted by 42. An image frame 44 representing the field of view 42, which is captured by the image detection device 22 shows the subject 12 which is in this case a human being to be measured. In the image frame 44, a grid 46 divides the image frame 44 in different portions and defines image sections 48 to distinguish different areas in the field of view 42 and to determine different motion vectors in the field of view 42. In order to determine the region of interest, i.e. the thorax 18 and the abdomen 20 of the subject 12, movement pattern are derived from each of the image sections 48 of the image frame 44 and the alternating signals S are determined from motion vectors determined from the movement pattern of each of the image sections 48 as described above. The motion vectors are determined by pattern detection or edge detection within the different image sections. On the basis of the frequency analysis as described above it is determined whether the movement pattern of the different image sections 48 correspond to a respiratory signal of the subject 12 in the field of view 42 or whether the movement pattern are disturbance signals or noise. The determination whether the movement pattern includes respiratory signals or not is performed on the basis of the spectral parameter and/or the spectral energy and e.g. whether the spectral energy in a frequency band is larger than a certain percentage of the entire spectral energy of the respective alternating signal.

On the basis of these data, which are determined for each of the image sections 48, the selection unit selects those image sections which include the respiration signals and may combine those selected image sections 48 to the region of interest, which is in FIG. 5 generally denoted by 50. The region of interest 50 shown in FIG. 5 comprises the two indicative portions 18, 20 corresponding to the thorax 18 and the abdomen 20. In a certain embodiment, the analysis unit 32 may determine different regions of interest which may be separated from each other in order to determine the different alternating signals from the different indicative portions 18, 20 of the subject 12.

On the basis of the different alternating signals S which are derived from the image sections 48 of the region of interest 50, the evaluation unit 34 determines the different respiration signals corresponding to the breathing motion of the thorax 18 and the abdomen 20. The analysis unit 32, in particular the selection unit of the analysis unit 32 determines a weight factor for each of the selected image sections 48 of the region of interest 50 in order to weight the alternating signals S of the different sections 48 on the basis of the signal quality. The weight factor determined by the analysis unit 32 may be calculated on the basis of the frequency how often the respective image section is selected by the selection unit. In other words, the alternating signals S from those image sections 48 which are selected more often as a selected image section 48 are given more weight and the image sections 48 selected less often are given less weight to calculate the respective respiratory signals.

The alternating signals S comprising identical or corresponding wave forms are combined (by the evaluation unit 32) to a single respiration signal since these alternating signals S are considered to be derived from the same indicative portion 18, 20. If the alternating signals from different sections 48 have a larger difference, e.g. phase shift, those alternating signals S are considered to be derived from different indicative portions 18, 20 and are not combined directly to one respiration signal. The combination steps are performed by the evaluation unit 32.

It is also possible to determine the respiration signals of each of the image sections 48 separately and to determine by means of the evaluation unit 34 a spatial respiration map of the subject 12 and the region of interest 50.

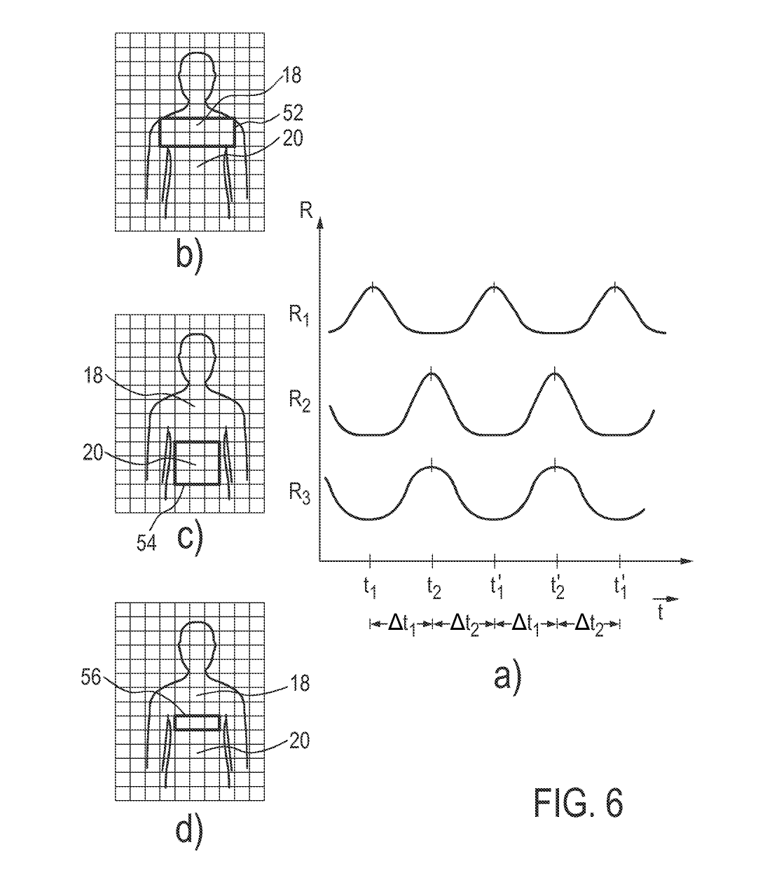

FIG. 6a shows a timing diagram comprising three different respiration signals R1, R2 and R3 which are derived by motion vector detection contactless from different portions of the subject 12. The regions of the subject 12 from which the respiration signals R1, R2, R3 are derived are schematically shown in the captured images of FIGS. 6b, c and d.

The first respiration signal R1 is determined from a region of interest 52 including the thorax 18 or the chest 18 as indicated in FIG. 6b. The second respiration signal R2 is derived from a region of interest 54 including the abdomen 20 or the belly 20 of the subject 12 as indicated in FIG. 6c. The second respiration signal R2 is phase shifted to the respiration signal R1 of the thorax 18. The third respiration signal R3 is determined from a region of interest 56 between the thorax 18 and the abdomen 20 of the subject 12 as shown in FIG. 6d. The third respiration signal R3 shows a respiration corresponding to the abdominal respiration of the second respiration signal R2, however, the third respiration signal R3 has a broader peak shape since the alternating signals derived from this intermediate portion do not have the signal strength as the thorax 18 and the abdomen 20.

The respiration signals R1 and R2, R3 have their peaks corresponding to the movement of the respective indicative portion 18, 20 at different points in time t1, t2 and are phase shifted to each other as indicated by Δt1 and Δt2. The phase shift Δt1, Δt2 corresponds to the alternating movement of the thorax 18 and the abdomen 20 due to the respiration of the subject 12. Hence, the different respiration signals R1, R2, R3 can be derived independently by means of the apparatus 10 contactless and remotely and additional information like the phase shift Δt1, Δt2 can be determined from the remote measurement.

On the basis of the additional information like the phase shift Δt1, Δt2 additional diagnostics can be performed in order to determine certain injuries of the subject 12.

In a certain embodiment, the phase shift Δt1, Δt2 of the respiration signals R1, R2, R3 is determined and a general respiration signal is determined by combining the different respiration signals R1, R2, R3 derived from the different regions of interest 52, 54, 56 indicative portions 18, 20 wherein the phase shift is considered and the signals are respectively shifted so that the respiration signals R1, R2, R3 are in phase before the signals are combined. By means of this combination, a reliable respiration signal can be determined even if the single respiration signals R1, R2, R3 have a poor signal strength.

In a simple embodiment of the invention, the image data is evaluated on the basis of the different rows of the grid 46 wherein one alternating signal S of one image section 48 of each of the rows is selected having the highest signal strength and the respective respiration signal R1, R2, R3 is determined for each of the rows on the basis of the one selected image section 48. This can reduce the technical effort of the apparatus 10 and the calculation time for determining the respiration signals R1, R2, R3.

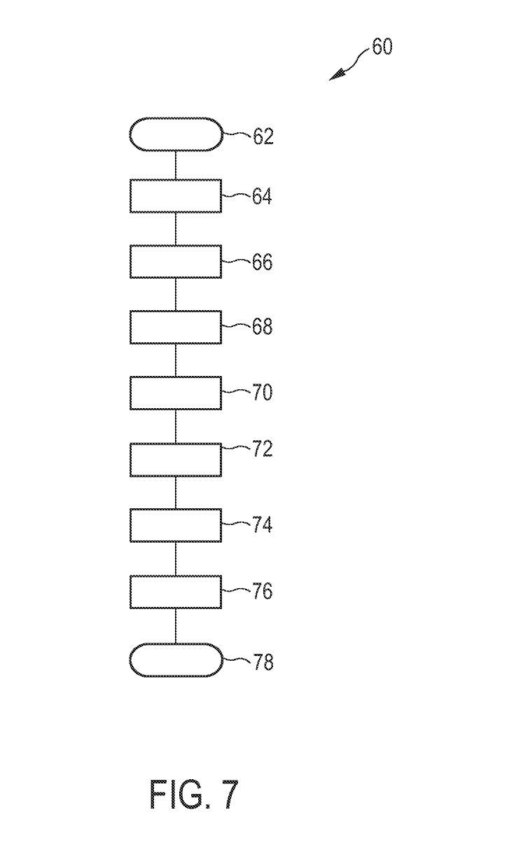

FIG. 7 shows a block diagram illustrating method steps to detect respiration signals from the subject 12. The method is generally denoted by 60. The method 60 starts with step 62. At step 64, an image frame 44 is detected by means of the image detection device 22. At step 66, the image frame 44 or the image data 26 is provided via the interface 28 to the image processing unit 30 and evaluated by the image processing unit 30 by means of pattern detection or edge detection and the motion vectors are determined for each of the image sections 48 as described above. Depending on the motion vectors, a corresponding alternating signal S is calculated for each of the image sections 48 at step 68. The alternating signals S are provided to the analysis unit 32 and the analysis unit 32 analyzes the alternating signals S at step 70. The analysis step 70 comprises the filtering of the alternating signals by means of the filter unit. At step 72, the selection unit selects those image sections 48 which comprises respiration signals of the subject 12 and the region of interest 50 is determined. At step 74, the evaluation unit 34 evaluates the alternating signals S received from the analysis unit 32 and determines the different respiration signals R1, R2, R3 of the subject 12 from the different indicative portions 18, 20.

At step 76, the different respiration signals R1, R2, R3 are displayed by means of the display 36.

At step 78, the method 60 ends. Hence the method 60 can determine different respiration signals R1, R2, R3 from the one subject 12 based on motion detection of the different indicative portions 18, 20.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or an does not exclude a plurality. A single element or other unit may fulfill the functions of several items recited in the claims. The mere fact that certain measures are recited in mutually different dependent claims does not indicate that a combination of these measures cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems.

Furthermore, the different embodiments can take the form of a computer program product accessible from a computer usable or computer readable medium providing program code for use by or in connection with a computer or any device or system that executes instructions. For the purposes of this disclosure, a computer usable or computer readable medium can generally be any tangible device or apparatus that can contain, store, communicate, propagate, or transport the program for use by or in connection with the instruction execution device.

In so far as embodiments of the disclosure have been described as being implemented, at least in part, by software-controlled data processing devices, it will be appreciated that the non-transitory machine-readable medium carrying such software, such as an optical disk, a magnetic disk, semiconductor memory or the like, is also considered to represent an embodiment of the present disclosure.

The computer usable or computer readable medium can be, for example, without limitation, an electronic, magnetic, optical, electromagnetic, infrared, or semiconductor system, or a propagation medium. Non-limiting examples of a computer readable medium include a semiconductor or solid state memory, magnetic tape, a removable computer diskette, a random access memory (RAM), a read-only memory (ROM), a rigid magnetic disk, and an optical disk. Optical disks may include compact disk-read only memory (CD-ROM), compact disk-read/write (CD-R/W), and DVD.

Further, a computer usable or computer readable medium may contain or store a computer readable or usable program code such that when the computer readable or usable program code is executed on a computer, the execution of this computer readable or usable program code causes the computer to transmit another computer readable or usable program code over a communications link. This communications link may use a medium that is, for example, without limitation, physical or wireless.

A data processing system or device suitable for storing and/or executing computer readable or computer usable program code will include one or more processors coupled directly or indirectly to memory elements through a communications fabric, such as a system bus. The memory elements may include local memory employed during actual execution of the program code, bulk storage, and cache memories, which provide temporary storage of at least some computer readable or computer usable program code to reduce the number of times code may be retrieved from bulk storage during execution of the code.

Input/output, or I/O devices, can be coupled to the system either directly or through intervening I/O controllers. These devices may include, for example, without limitation, keyboards, touch screen displays, and pointing devices. Different communications adapters may also be coupled to the system to enable the data processing system to become coupled to other data processing systems, remote printers, or storage devices through intervening private or public networks. Non-limiting examples are modems and network adapters and are just a few of the currently available types of communications adapters.

The description of the different illustrative embodiments has been presented for purposes of illustration and description and is not intended to be exhaustive or limited to the embodiments in the form disclosed. Many modifications and variations will be apparent to those of ordinary skill in the art. Further, different illustrative embodiments may provide different advantages as compared to other illustrative embodiments. The embodiment or embodiments selected are chosen and described in order to best explain the principles of the embodiments, the practical application, and to enable others of ordinary skill in the art to understand the disclosure for various embodiments with various modifications as are suited to the particular use contemplated. Other variations to the disclosed embodiments can be understood and effected by those skilled in the art in practicing the claimed invention, from a study of the drawings, the disclosure, and the appended claims.

1. An apparatus for determining respiration signals from a subject, comprising:
a receiving unit that receives image data determined from the subject in a field of view,
a processing unit that evaluates the image data, wherein the processing unit is adapted to determine a plurality of different alternating signals corresponding to vital sign information of the subject from a plurality of different areas of the field of view on the basis of movement pattern, and
an evaluation unit that evaluates the different alternating signals and that determines a plurality of different respiration signals from the subject on the basis of the different alternating signals determined from the different areas of the field of view.

2. The apparatus as claimed in claim 1, wherein the processing unit is adapted to define a plurality of image sections in the image data and to determine one alternating signal corresponding to the vital sign information from each of the image sections on the basis of movement pattern detection.

3. The apparatus as claimed in claim 2, wherein the processing unit is adapted to define the different image sections as an array of image sections in the image data.

4. The apparatus as claimed in claim 2, further comprising a frequency analysis unit that determines spectral parameter of the alternating signals determined from the different image sections, and a selection unit that selects different image sections on the basis of the spectral parameter as the different areas to determine the different respiration signals.

5. The apparatus as claimed in claim 4, wherein the spectral parameter is a spectral energy of the alternating signals.

6. The apparatus as claimed in claim 5, wherein the selection unit is adapted to select the image sections if the spectral energy of a predefined frequency band of the alternating signals exceeds a threshold level.

7. The apparatus as claimed in claim 1, wherein the different respiration signals are determined on the basis of motion vectors derived from different portions of the subject.

8. The apparatus as claimed in claim 1, wherein the different respiration signals are time dependent alternating signals having different waveforms.

9. The apparatus as claimed in claim 1, wherein the different respiration signals are time dependent signals having a phase shift to each other.

10. The apparatus as claimed in claim 1, wherein the evaluation unit is adapted to determine a signal difference of the different respiration signals as additional respiration information of the subject.

11. The apparatus as claimed in claim 9, wherein the evaluation unit is adapted to determine the phase shift of the different respiration signals and to combine the different respirations signals to one general respiration signal considering the determined phase shift.

12. The apparatus as claimed in claim 1, wherein the evaluation unit is adapted to determine an array of respiration signals on the basis of the different respiration signals derived from the different image sections to provide a spatial respiration map of the subject.

13. The apparatus as claimed in claim 4, wherein the selection unit is adapted to determine a weight factor for each of the selected different image sections and wherein the evaluation unit is adapted to determine the different respiration signals on the basis of the alternating signals of the selected image sections weight by means of the respective weight factor.

14. The apparatus as claimed in claim 13, wherein the selection unit is adapted to perform the selection on a regular basis and wherein the weight factor for each of the selected image section is determined on the basis of a frequency of selection of the respective image section.

15. A method for determining respiration signals from a subject, comprising the steps of:
receiving image data determined from the subject in a field of view,
evaluating the image data,
determining a plurality of different alternating signals corresponding to vital sign information of the subject from different areas of the field of view on the basis of movement pattern,
evaluating the different alternating signals, and
determining a plurality of different respiration signals from the subject on the basis of the different alternating signals determined from different areas of the field of view.

16. A computer readable non-transitory medium having instructions stored thereon which, when carried out on a computer, cause the computer to perform the following steps of the method as claimed in claim 15.

17. An apparatus for determining respiration signals from a subject, comprising:
a receiving unit that receives image data determined from the subject in a field of view,
a processing unit that defines a plurality of image sections in the image data and that determines one alternating signal corresponding to the vital sign information from each of the image sections on the basis of movement pattern detection, wherein the processing unit is adapted to determine the alternating signals corresponding to the vital sign information of the subject from different image sections of the field of view on the basis of movement pattern, and
an evaluation unit that evaluates the different alternating signals and that determines a plurality of different respiration signals on the basis of the movement pattern derived from different portions of the subject and the different alternating signals determined from the different areas of the field of view.
